(19) United States
(12) Patent Application Publication
(10) Pub. No.: US 2014/0335625 A1
(43) Pub. Date: Nov. 13, 2014

(54) Temperature Control Method in a Laboratory Scale Reactor

(71) Applicant: Randal L. Hatfield, Port Hueneme, CA (US)

(72) Inventor: Randal L. Hatfield, Port Hueneme, CA (US)

(73) Assignee: CDTi, Ventura, CA (US)

(21) Appl. No.: 13/891,745

(22) Filed: May 10, 2013

(51) Int. Cl. G01N 33/00

(52) U.S. Cl. CPC G01N 33/0016
USPC 436/37; 422/83

(57) ABSTRACT

Disclosed herein is a method of separating variations in the mass flow of gas through a catalyst from the thermal load observed by the temperature control system in a test bench. The method may include separating the temperature control component from the mass flow control component.

### CROSS-REFERENCE TO RELATED APPLICATIONS

N/A

### BACKGROUND

1. Field of the Disclosure

The present disclosure relates to a laboratory test device and, more particularly, to a method for controlling test gas temperatures in a test bench.

2. Background Information

Catalysts may need to be tested to evaluate their performance and their response to parameter changes. Devices of use in testing catalysts may include one or more combustion engines; however, the use of these engines may be expensive, require higher maintenance than desired, and be more time consuming. Additionally, the use of these engines may not allow individual parameter variations or calibrations of use when testing catalysts. Other test devices suitable for testing catalysts may include Laboratory Scale Reactors, commonly referred to as Test Benches, and may allow a greater control over the testing conditions of the catalyst.

However, Laboratory-scale reactors may experience difficulties in separating control of one or more individual parameters or calibrations, including the separation of control of mass flow through the sample from temperature control of the gas flowing through the sample. This may limit the conditions laboratory scale reactors may produce for testing suitable materials.

As such, there is a continuing need for improvements in test devices so as to allow a greater range of testing conditions.

### SUMMARY

The present disclosure may include a method for separating temperature control and mass flow control in a test bench of use in testing catalysts.

The method may include isolating the thermal load perceived by the heating elements from the variation of the gas flow perceived by the catalyst being tested, where excess gas may undergo any suitable venting, including venting over a catalyst holder, venting to a confined environment, venting to the general environment, or any suitable combination. This may allow the space-velocity of gas processed by the heater to vary independently from the space-velocity of the gas flowing through the sample.

Numerous other aspects, features and advantages of the present disclosure may be made apparent from the following detailed description, taken together with the drawing figures.

### BRIEF DESCRIPTION OF THE DRAWINGS

These and further features, aspects and advantages of the embodiments of the present disclosure will be apparent with regard to the following description, appended claims and accompanying drawings where:

FIG. 1 is a flow chart of a method for separating mass flow control from temperature control in a laboratory scale reactor.

FIG. 2 illustrates a method for controlling temperature and mass flow through a sample in a laboratory scale reactor.

It should be understood that these drawings are not necessarily to scale and they can illustrate a simplified representation of the preferred features of the embodiments of the present disclosure.

### DETAILED DESCRIPTION

### Definitions

As used here, the following terms have the following definitions:

Mass flow controller (MFC) refers to any computer controlled analog or digital device of use in controlling the flow rate of fluids and/or gases.

Temperature controller refers to any device of use in controlling temperature in a process.

Laboratory Scale Reactor/Test Bench refers to any apparatus suitable for testing a material with a test gas.

Oxidizing agent refers to any substance that may take electrons from another substance in a redox chemical reaction.

Reducing agents refers to any substance that may give electrons to another substance in a redox chemical reaction.

Gas mixture refers to the mixture obtained from combining oxidizing agents, reducing agents, inert gases, or any other suitable gases.

Water-gas mixture refers to the mixture obtained from combining water vapor with a gas mixture.

Test Gas refers to any gas mixture of use in chemically testing an interaction between it and one or more materials.

Catalyst refers to one or more materials that may be of use in the conversion of one or more other materials.

The description of the drawings, as follows, illustrates the general principles of the present disclosure with reference to various alternatives and embodiments. The present disclosure may, however, be embodied in different forms and should not be limited to the embodiments here referred. Suitable embodiments for other applications will be apparent to those skilled in the art.

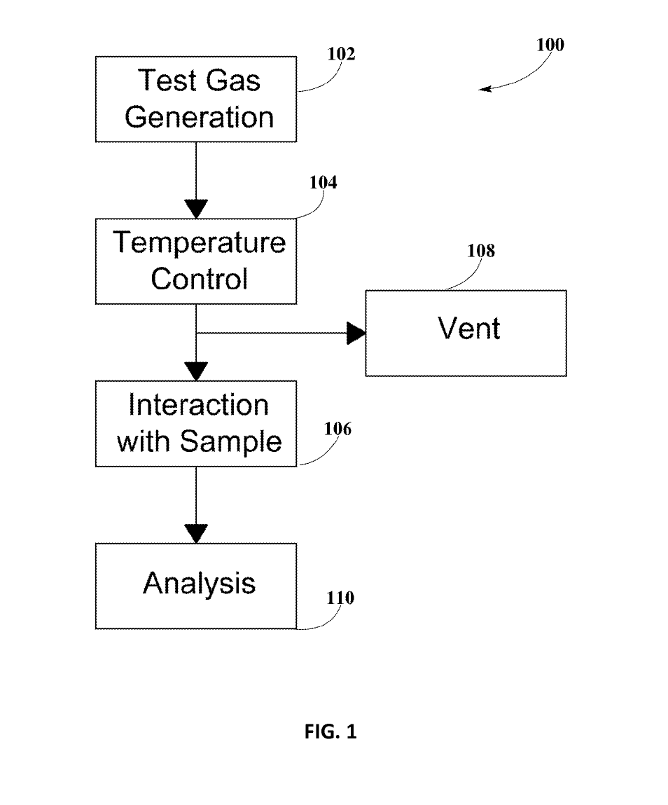

FIG. 1 is a flowchart of a method for testing a material in a Laboratory Scale Reactor. In Testing Method 100, a suitable test gas may be generated in Test Gas Generation 102. The test gas may then be heated to any suitable temperature in Temperature Control 104. Any suitable portion of test gas heated in Temperature Control 104 may then undergo Interaction with Sample 106, where any portion not undergoing Interaction with Sample 106 may undergo any suitable venting in Vent 108. Any portion having undergone Interaction with Sample 106 may then undergo any suitable Analysis 110.

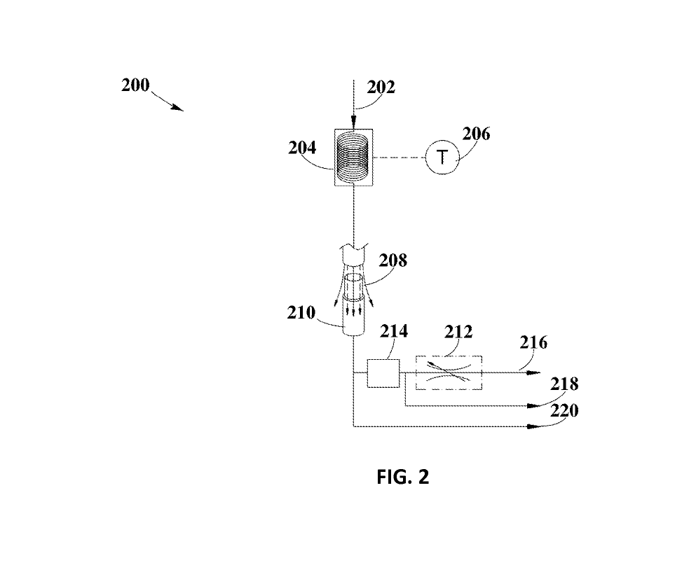

FIG. 2 shows Temperature and Flow Control Method 200, having Input 202, Heater 204, Temperature Controller 206, Catalyst Sample 208, Catalyst Holder 210, Mass Flow Controller 212, Pre-treatment Device 214, and Output 216.

Input 202 may provide any suitable test gas to Temperature and Flow Control Method 200, where gas flowing from Input 202 may then be heated in Heater 204. Heater 204 may be any suitable heating device, including a serpentine heater, which may be controlled by any suitable Temperature Controller 206, including thermocouples, thermistors, or any suitable combination thereof.

Any suitable portion of test gas heated by Heater 204 may then flow through Catalyst Sample 208 held by Catalyst Holder 210, where Catalyst Sample 208 may be any material suitable for being tested with test gas provided by Input 202. Any suitable portion of test gas not flowing through Catalyst Sample 208 may be vented in any suitable way, including venting through Catalyst Holder 210 and venting to the environment.

Any suitable portion of test gas flowing through Catalyst Sample 208 may be controlled by any number of suitable Mass Flow Controllers 212, where any the flow between Catalyst Sample 208 and Mass Flow Controllers 212 may undergo treatment in one or more suitable Pre-treatment Devices 214, where suitable devices may include heat blocks and cooling baths. Any portion of test gas flowing through one or more Mass Flow Controllers 212 may then exit the control system through one or more Outputs 216, where the portion may then undergo any suitable Analysis 110. Suitable analyses may include Flame Ionization Detection, NOx detection, CO detection, Hydrocarbon detection, Fourier Transform Infrared Spectroscopy (FTIR) and any suitable combination thereof, where suitable analyses may include any suitable treatments required to perform the analyses.

Any suitable portion of test gas flowing through Catalyst Sample 208 and Pre-treatment devices 214 not flowing through Mass Flow Controllers 212 may exit the control system through one or more Outputs 218, where the portion may then undergo any suitable Analysis 110. Suitable analyses may include Flame Ionization Detection, NOx detection, CO detection, Hydrocarbon detection, Fourier Transform Infrared Spectroscopy (FTIR) and any suitable combination thereof, where suitable analyses may include any suitable treatments required to perform the analyses.

Any suitable portion of test gas flowing through Catalyst Sample 208 not flowing through Pre-Treatment Devices 214 may exit the control system through one or more Outputs 220, where the portion may then undergo any suitable Analysis 110. Suitable analyses may include Flame Ionization Detection, NOx detection, CO detection, Hydrocarbon detection, Fourier Transform Infrared Spectroscopy (FTIR), and any suitable combination thereof, where suitable analyses may include any suitable treatments required to perform the analyses.

What is claimed is:

1. An apparatus for analyzing a fluid, comprising:
a heating chamber comprising at least one heating element suitable for heating the fluid to a heating temperature and for imparting a first space velocity to the fluid;
a heating controller suitable for controlling the heating temperature of the fluid;
at least one catalyst sample provided substantially in-line following the heating chamber and suitable for interacting with a first portion of the fluid having a second space velocity;
at least one vent suitable for venting, prior to interacting with the catalyst sample, of a second portion of the fluid, thereby imparting the second space velocity to the first portion of the fluid;
at least one mass flow controller for controlling flow of a mass-flow controlled one of the interacted first portion of the fluid; and
at least three outputs, comprising:
a first output suitable for outputting the mass flow controlled one of the interacted first portion;
a second output suitable for substantially directly outputting a pre-treated one of the interacted first portion; and
a third output suitable for substantially directly outputting the interacted first portion.

2. The apparatus of claim 1, further comprising a calayst holder suitable for receiving the interacted first portion substantially directly from the catalyst sample.

3. The apparatus of claim 1, wherein the first space velocity is not equal to the second space velocity.

4. The apparatus of claim 1, wherein the at least one fluid comprises gas.

5. The apparatus of claim 1, wherein the heating controller controls a rate of the heating.

6. The apparatus of claim 1, wherein the heating controller comprises one selected from the group consisting of a thermocouple, thermistor, and any combination thereof.

7. The apparatus of claim 1, wherein the heating element comprises a serpentine heater.

8. The appartus of claim 1, further comprising a pre-treatment device suitable for providing the pre-treated one of the interacted first portion.

9. The apparatus of claim 8, wherein the pre-treatment device comprises one selected from the group consisting of a heat block, cooling bath, and combinations thereof.

10. The apparatus of claim 1, further comprising at least one analyzer suitable for analyzing the fluid output at at least one of the at least three outputs.

11. The apparatus of claim 10, wherein the analyzing comprises one selected from the group consisting of flame ionization detection, CO detection, hydrocarbon detection, fourier transform infrared detection, and combinations thereof.

12. A method for analyzing at least one fluid, comprising:
heating at least one fluid in a heating chamber, the heating chamber comprising at least one heating element and at least one heating control for controlling a heating temperature of the at least one fluid;
venting at least a second portion of the at least one fluid after said heating;
interacting a first, non-vented portion of the at least one fluid with a catalyst after said venting;
controlling a flow of the interacted first, non-vented portion of the at least one fluid utilizing at least one mass flow controller;
outputting at least a flow contolled one of the interacted first, non-vented portion of the at least one fluid, and a non-flow controlled one of the interacted first, non-vented portion of the at least one fluid.

13. The method of claim 12, wherein, prior to said venting, the at least one fluid comprises a first space velocity, and wherein, following said venting, the at least one fluid comprises a second space velocity, and wherein the first space velocity is unequal to the second space velocity.

14. The apparatus of claim 12, wherein the at least one fluid comprises gas.

15. The method of claim 12, further comprising controlling a rate of the heating via the heating control.

16. The method of claim 12, wherein the heating element comprises a serpentine heater.

17. The method of claim 12, further comprising pre-treating the interacted first, non-vented portion using a pre-treatment device.

18. The method of claim 17, wherein the pre-treatment device comprises one selected from the group consisting of a heat block, cooling bath, and combinations thereof.

19. The method of claim 12, further comprising analyzing at least one of the outputted interacted first, non-vented portions of the at least one fluid.

20. The method of claim 19, wherein the analyzing comprises one selected from the group consisting of flame ionization detection, CO detection, hydrocarbon detection, fourier transform infrared detection, and combinations thereof.
